(19) United States
(12) Patent Application Publication
(10) Pub. No.: US 2012/0219529 A1
(43) Pub. Date: Aug. 30, 2012

(54) Silver ion compositions of pectins

(75) Inventor: George H. Scherr, Park Forest, IL (US)

(73) Assignee: George H. Scherr Trust

(21) Appl. No.: 12/932,486

(22) Filed: Feb. 28, 2011

(51) Int. Cl. A61K 31/738; C08B 37/06; A61P 31/04; A61P 31/10; B05D 5/00; A61P 31/00; A61P 19/00; A61P 43/00; A61K 35/12; A61K 35/00; A61P 31/12

(52) U.S. Cl. 424/93.7; 514/54; 424/93.1; 536/2; 427/2.3; 427/2.24

(57) ABSTRACT

The invention described herein relates to the preparation of silver compounds prepared from pectin and pectin derivatives and the process for preparing them. The silver derivatives thus prepared are amenable for use in the preparation of medical and/or veterinary dressings or implants.

### DESCRIPTION OF THE PRIOR ART

The increase in longevity of the population in the United States and the concomitant increase in the requirements for implantable devices such as pacemakers, or prosthetics to repair fractures has been accompanied by an increase in infections following the related implantations in patients. Thus, estimates conducted by market research professionals indicate that for more than the approximate 400,000 pacemakers implanted each year in the United States, 5.82% of them result in infections that require prolonged therapy or repeated surgical procedures. Consequently, estimates reported in the New England Journal of Medicine (2004: 350: 1422-9) have indicated that of the 2,000,000 cases of nosocomial infections that occur each year in the United States, approximately half of them are associated with the use of indwelling devices such as pacemakers.

In a study undertaken by Blom et al. “Infection after total knee arthroplasty” (TKA) it was shown that infection following knee arthroplasty occurred at a rate between 0.5% and 12% depending upon the specific surgery reported and the individuals involved.

One of the significant factors employed in this study was the use of 0.05% chlorhexidine lavage in these patients that were subjected to the arthroplasty surgery. The authors also reported that “the outcome of patients with deep infections after primary TKA was poor in that only two of nine (22.2%) patients retained the prosthesis free from infection.”

It is not unknown that organisms, especially Staphylococcus organisms, can become resistant to chlorhexidine, but what may be as significant is the fact that chlorhexidine has been 2 shown to result in hypersensitivity in many patients and that a number of patients have had induced anaphylactic shock. In fact, the severity of such cases subjected to dressings containing chlorhexidine applied to open wounds has resulted in the United States Food and Drug Administration barring the use of chlorhexidine-impregnated dressings from use on infants.

TyRx Pharma, Inc. has received approval from the United States Food and Drug Administration by way of a 510(k) Number K063091 for a pacemaker or a defibrillator. The defibrillator is covered with filaments of polypropylene and the polypropylene is coated with a polyarylate polymer which can be absorbed by the body in approximately 140 days. The polyarylate polymer contains two antimicrobial agents, rifampin and minocycline in concentrations of 86 μg/cm².

Though the two antimicrobial agents have been reported showing antimicrobial activity against a number of bacteria that may cause infection following surgical implantation of pacemakers or defibrillators, the studies have demonstrated that the in vitro as well as the in vivo activity of the two antimicrobials are variable against non-epidermidis strains of coagulase-negative staphylococci. Assuming the validity of the New England Journal of Medicine data cited above, it should be expected that of the approximate one million cases of nosocomial infections resulting from indwelling devices such as pacemakers and of these it is not known how many patients are found to be infected with non-epidermidis strains of coagulase-negative staphylococci. In addition, other pathogens which may cause infections after insertion of pacemakers or defibrillators also can become resistant to the antibiotics for rifampin and minocycline.

Since the report of the 510(k) K063091 indicates that the polyarylate polymer containing the two antimicrobial agents takes approximately 140 days to be absorbed, the 86 μg/cm² of the two antibiotics which are contained in the polyarylate polymer during this period may release an amount of antibiotic over a period of 140 days that would be too low to cause a complete biocidal effect for the infectious organisms.

Howmedica International S. de R. L. in Limerick, Ireland, manufactures a bone cement containing the antibiotic tobramycin which is used in hip and knee replacements. It has been estimated that there are approximately 640,000 hip and knee replacements performed in the United States every year. Many of these hip and knee replacements will use a bone cement containing the antibiotic tobramycin. Antibiotic resistance to tobramycin has been well-documented in innumerable publications such as the report by J. C. Nickel et al. “Tobramycin resistance of Pseudomonas aeruginosa cells growing as a biofilm on urinary catheter material” published in Antimicrobial Agents Chemotherapy 1985 April; 27(4):619-24, and also in the report by Hosmin Anwar et al. “Tobramycin resistance of mucoid Pseudomonas aeruginosa biofilm grown under iron limitation” published in the Journal of Antimicrobial Chemotherapy (1989) 24, 647-655.

Moojen, et al. (J. Arthroplasty. 2008 Dec;23(8):1152-6. Epub 2008 Mar. 4) reported the antibiotic release during a six week period in patients who had been treated with a hip spacer containing an antibiotic bone cement. All of the patients showed a burst release of an antibiotic during the first week, but the spacers showed very little additional release after the first week. The authors therefore, concluded that “ . . . one should be cautious toward using low-dose antibiotic bone cement for spacers because this could result in an unsuccessful eradication of infection.”

Minelli, et al. (J. Antimicrob. Chemother. 2004 August; 54(2):570; author reply 570-1) reported on the delivery of the two antibiotics gentamicin and vancomycin which had been placed in polymethylmethacrylate spacers during their implantation for the treatment of total hip replacement infections. Twenty such spacers which utilized 1.9% gentamicin and 2.5% vancomycin mixed with the polymethylmethacrylate cement were used to fill holes drilled in the cement of 14 of the 20 spacers just prior to implantation. These antibiotic concentrations are to be considered relatively high. Examination at 3-6 months after implantation of the concentrations of the antibiotics in the cement showed that there was a high initial release of the drugs and a reduced, but constant, release of the drugs over the next few days. In the twenty patients studied with these antibiotic-impregnated cements in hip replacement spacers, at the end of six months it was found that the range of gentamicin still present in the cement ranged from 0.05%-0.4% or an approximate range of ten-fold difference among the twenty patients examined. For the vancomycin, at the end of a six-month period, the concentration of the vancomycin found in the spacers ranged from 0.8%-3.3% or an approximate four-fold difference among the twenty patients that were examined.

A paper by Illingworth, et al. (J. Heart Valve Dis.1998 September; 7(5):524-30) reported on a silver metal coated polyester fabric in the form of a cuff utilized for the implantation of mechanical hearts and the ability of the silver cuff to inhibit the pathogen S. epidermidis. The ability to show inhibition for this heart valve device has been approved by the United States Food and Drug Administration under their 510(k) Number K000119.

It has been generally accepted that the olygodynamic action of silver would require that the silver metal be converted to a silver ion as has been reported in numerous reports: The effect of various metals on biological processes has generally been referred to as an 5 olygodynamic action. A detailed discussion of the history of such olygodynamic action with particular emphasis on the use of silver is contained in Chapters 24 and 28 of the monograph by Lawrence and Block, Disinfection, Sterilization, and Preservation, Lea and Febiger, Philadelphia, 1968.

Studies examining the mechanism of action by silver in demonstrating antimicrobial activity led various workers to conclude that the presence of silver ions was more important than the amount of silver metal or the time that the substrate containing microbes was exposed to the silver. Silver was prepared in the form of a spongy metallic form or by coating various products which contain large surfaces such as sand by K. Süpfle and R. Werner (1951 Microdosimetric investigation of the oligodynamic effect of silver. Mikrochemie ver. Mikrochim. Acta., 36/37, 866-881). These workers showed that E. coli placed in flasks having counts of 18,000 E. coli per ml of water, when such counts were exposed to flasks containing sand coated with silver at a concentration of 10% silver of the amount of sand, would result in a sterile environment in four hours. Even counts as high as 120,000 E. coli per ml of solution, resulted in sterility in 24 hours. The relatively low count of E. Coli of 1.8×10³ and the very high concentration of silver coated onto the sand of 10% would readily explain a sterile environment in four hours. However, in vivo experiments reports in 510(k) reports that are available have never utilized a silver concentration as high as 10% which could be toxic to a patient and result in argyria.

A singular aspect of the chemical activity of silver and silver salts has to do with the ease with which silver ions combine with proteins, oxygen, and various halogens to result in insoluble material. Thus for example, oxides of silver (as Ag₂O or Ag O) are considered insoluble in water as is silver chloride. L. Goodman and A. Gilman (The Pharmacological Basis of Therapeutics, 3^{rd} Ed. New York, The Macmillan Company 1965) reported that the toxic effects of silver compounds on microorganisms is probably due to the silver ions which precipitate the protein of bacterial protoplasm. It is very well known that soluble silver salts such as silver nitrate will quickly precipitate protein and oxidize to a dark brown or black precipitate. The silver protein complex so formed contributes to a sustained antimicrobial action by slowly liberating small amounts of silver ions. It therefore would be necessary for the silver metal, in the report by Illingworth, et al. for the device in repairing a diseased or damaged heart valve, to be ionized to the silver ionic state.

Neither silver ions nor silver colloids should be released into a wound in a relatively large amount in a short period of time; otherwise the patient will turn blue as a result of silver poisoning (argyria) (Trop et al., 2006).

Azary Technologies LLC has produced a silver-containing fabric in which a nylon-containing textile fiber which contains silver which has been plated onto the nylon. The Azary company in submitting data pertinent to their United States Food and Drug Administration 510(k) device, K040518, has indicated that the silver contained on the nylon fiber is pure elementary silver at a purity of 99.9%. These textiles coated with elemental silver have been approved for coating nylon which can be used in a medical dressing or in certain wearing apparel such as socks. Here too, it is expected that the elemental silver will have to be converted to ionic silver by some mechanism in order to ensure antimicrobial activity.

RyMed Technologies, Inc. in their 510(k) K093489 has produced a catheter implant containing silver ions and chlorhexidine which are intended to inhibit the growth of microorganisms on the treated surfaces of the device, which include the septum and the fluid 7 path. The subject device is not intended to treat existing infections. The device is not intended to have any effect on contaminated infusion solutions.

A report by Scardillo in the publication Infection Control Resource, presents the various sources of surgical site and other sources of infections and their treatment (Infection Control Resource, Vol. 2 No.1, pp. 2-7—Saxe Healthcare Communications).

Rochester Medical has introduced with the approval of the United States Food and Drug Administration (FDA 510(k) Number K033477) a series of Foley Catheters which are considered implants. These catheters are manufactured entirely from a silicone. The silicone matrix contains a nitrofurazone antimicrobial agent. Test results for toxicity published by the Rochester Medical Corporation as part of their 510(k) notification indicates that “Test extracts showed a cytotoxic effect. The antibacterial agent is a known mutagen.” Any antimicrobial agent that is known to be a mutagen may well be considered undesirable for use in implants since many mutagens are also carcinogens. Mutagens that are shown to be carcinogenic cannot be utilized in drug preparations in drug or medical device applications as has been mandated by the Congress of the United States.

The development of calcium alginate fibers by Courtaulds in Coventry, England, principally for the textile industry, prompted the investigation by researchers such as Scherr to prepare medical dressings from the calcium alginate fibers, especially in the light of their hemostatic activity and slight antimicrobial activity. (See U.S. Pat. No. 5,674,524, issued Oct. 7, 1997, entitled Alginate Fibrous Dressings and Method of Making; U.S. Pat. No. 5,718,916, issued Feb. 17, 1998, entitled Alginate Foam Products; U.S. Pat. No. 7,128,929, issued Oct. 31, 2006, entitled Alginate Foam Products.)

The increased interest in calcium alginate fiber dressings for the treatment of wounds further resulted in the incorporation of antimicrobial agents in such dressings. The resultant development by Scherr of a silver alginate moiety introduced antimicrobial activity for medical dressings that had a number of advantages over antibiotics. (See U.S. Pat. No. 6,696,077, issued Feb. 24, 2004, entitled Silver Alginate Foam Compositions; India Patent Number 221859, issued Jul. 8, 2008, entitled Alginate Foam Compositions; United Kingdom Patent. Number GB 2,357,765, issued Apr. 21, 2004, entitled Alginate Foam Compositions; Russian Patent Number 2322266, issued Feb. 18, 2003, entitled Alginate Foam Compositions.)

Aqueous insoluble salts are readily achieved with salts of calcium, aluminum, zinc, copper, iron, and silver, in which case, the insoluble alginate salt is suspended in an aqueous gel. A detailed discussion of the properties of alginates and the preparation of their solid states has been published by McDowell in his book, Properties of Alginates.

Pectins, either alone or in conjunction with alginates have also been used in the preparation of products that are amenable to the manufacture of wound dressings. Pectins are polysaccharides that contain a 1,4-linked α-D-galactosyluronic acid residue. There are essentially three pectic polysaccharides that are isolated from primary cell walls and they can be characterized as:
1. Homogalacturonans
2. Substituted galacturonans
3. Rhamnogalacturonans

Pectins may be esterified with methanol and the pectins can be classified as high- or low-ester pectins. The low-ester pectins usually require calcium to form a gel. A detailed 9 presentation of the chemistry of pectins has been published by the American Chemical Society and edited by Marshall L. Fishman and Joseph J. Jen (Chemistry and Function of Pectins, 1986).

Christian Bannert describes the preparation of a gel that can be utilized as a dressing into which gel disinfectants and medications can be added for treating the mucousa. These gels are obtained by using alginates which can be treated with a calcium salt. They can also be mixed with a pectin which has a low degree of esterification and would also be precipitable with a calcium salt. (See U.S. Pat. No. 5,147,648, issued Sep. 15, 1992).

U.S. Pat. No. 5,688,923 issued Nov. 18, 1997, describes a process of making pectin fibers that can be utilized in wound dressings.

U.S. Pat. No. 3,639,575 issued February 1972 to Irving R. Schmolka is concerned with the treatment of burns utilizing water soluble silver salts and a matrix for those silver salts which are composed of aqueous gels of polyoxyethylene polyoxypropylene block copolymers.

All seven claims of the Schmolka patent require that the composition prepared is designed “ . . . to treat a burn wound.” A silver water soluble salt may be used which can be silver nitrate, silver sulfate, silver acetate, and silver lactate monohydrate; these silver salts must dissolve in water at a minimum concentration 0.1 per cent.

The gels as prepared in the examples cited in U.S. Pat. No. 3,639,575 are transparent and form a clear gel at room temperature. When the gels are cooled, ostensibly below the temperature of room temperature, they become liquids.

The independent claim 1, upon which claims 2-6 of this patent are dependent, specifically sets forth that the matrix consists of a polyoxyethylene polyoxypropylene block copolymer having the formula HO(C₂H₄O)_{b}(C₃H₆O)ₐ(C₂H₄O)_{b}H wherein a is an integer such that the hydrophobe represented by (C₃H₆O) has a molecular weight of at least 2,250, and b is an integer such that the hydrophile portion represented by (C₂H₄O) constitutes from about 10 to 90 weight percent of the copolymer. U.S. Pat. No. 3,639,575 also sets forth that “A more detailed explanation of the preparation of these block copolymers may be found in U.S. Pat. No. 2,674,619 issued to Lester G. Lundsted on Apr. 6, 1954

U.S. Pat. No. 4,184,974 issued Jan. 22, 1980, to James W. Van Leuven, describes a liquid biocidal agent that has bacteriostatic and viricidal properties. The patent consists of an independent claims 1 and 4 claims that are all dependent on claim 1. Claim 1 provides that sterile distilled water be used and that such sterile distilled water should be “substantially free of alkaline earth cations, halide ions and strong acid anions. We have no way of knowing how much alkaline earth cations or halide ions or strong acid anions can exist in the sterile distilled water which can be still be characterized as being substantially free of these ingredients. It is well known that alkaline earth cations and especially halide ions are particularly reactive with silver ions to form highly insoluble compounds when silver ions are present in such distilled water, which amount might be considered substantially free and acceptable, but not completely free of alkaline earth cations.

The biocidal composition sodium polypectate described in James W. Van Leuven's U.S. Pat. No. 4,184,974 is used in the range of 100 to 400 parts per million, also includes in claim 1, a silver ion in the range from about 13 to 250 parts per million, and glycerin in the range of about 4 to 10% by weight; in addition, the pH of the water soluble base should be in the range of 7.2 to 7.8.

The inventor submits that the sodium polypeptide may be prepared by treating pectin with sodium carbonate in order to solubilized the pectins. The inventor therefore characterizes pectin as a water insoluble compound which is inconsistent with data submitted by innumerable documents that describe the utilization of pectins. The American Chemical Society monograph, Chemistry and Function of Pectins, unequivocally states that “Pectins are soluble in pure water.” The only caveat the ACS monograph submits is the case where “ . . . they (pectins) are insoluble in aqueous solutions in which they would gel at the same temperature if dissolved at a higher temperature.” However, that characterization for pectins when they become insoluble, is totally irrelevant to the specification and claims of U.S. Pat. No. 4,184,974. The inventor of this patent also submits that the preparation of sodium polypectate in order to solubilize the pectin with sodium carbonate also serves to result in the polypectate chelating readily with the alkaline earth cations such as calcium and magnesium. The reaction of calcium with pectin is well known and has been described in innumerable documents. The American Chemical Society's Symposium on pectins cited above also characterizes on page 8 that pectin will gel in the presence of divalent cations and increasing the concentration of divalent cations, such as calcium, increases the gelling temperature and gel strength.

In the patent by Gerrish, et al. (U.S. Pat. No. 5,688,923 issued Nov. 18, 1997), the inventors show that pectin can readily be dissolved in water as is indicated for example, in their claim 29. Their claim 35 indicates pectins will react with polyvalent cations wherein the cation which may consist of calcium, copper, barium, magnesium, zinc, and iron and will precipitate as an insoluble fiber of a pectate. The inventors have made no claim nor conducted any experiments as described in their U.S. Pat. No. 5,688,923 concerning the ability of silver ions to precipitate pectins. Also, they claim in their claim 6, that their claim 1 indicates they have non-pectin polysaccharides added to it such as hyaluronic acid, carrageenan, alginic acid, or sodium alginate. Alginic acid is insoluble in water, as has been well described in the chemical literature, but sodium alginate when added to the pectin where a fiber is to be made will certainly enhance the strength of the fiber.

Consequently, it is not clear and inconsistent with known chemistry why U.S. Pat. No. 4,184,974 of Van Leuven finds it necessary to solubilize pectins, which are known to be soluble in water, and that the preparation of a sodium polypectate is deemed necessary in order for the pectin to chelate with alkaline earth cations such as calcium when pectins readily chelate with calcium and become insoluble as has been well described in the literature.

The seventh edition of The Merck Index indicates that pectin is “completely soluble in 20 parts of water forming a viscous solution containing negatively charged hydrated particles.” For use as a medical product, pectin per se, as a powder was recommended in The Merck Index for hemostatic effect and as a paste for treatment of decubitus ulcers.

The MSDS sheet of Spectrum Laboratory Products, Inc. Gardena, Calif., 90248 dated Sep. 13, 2006 essentially paraphrases the data for pectin that The Merck Index cited above as being soluble in 20 parts of water and dissolves more readily in water if it is first moistened with alcohol or glycerol. The synonyms for pectin manufactured by Spectrum Laboratory Products, Inc. indicates that it may be also cited as Methoxypectin, Methyl pectin, Methyl pectinate, Pectinate, Pectrinic acid, Pectins, and Colyer Pectin. Chapter 5 in the book by A. Nussinovitch provides an excellent review of the chemistry of pectins, their preparation, and their reactions.

### SUMMARY OF THE INVENTION

The present invention relates to a preparation which comprises a silver pectate moiety which silver pectate moiety is insoluble in an aqueous solution. The pectin utilized should also be amenable to react with a soluble calcium salt and produce an insoluble calcium pectate in which the calcium aqueous soluble salt may be calcium chloride or calcium gluconate. The purpose of including a calcium pectate chemical impedance within the preparation is to serve as a chemical impedance to the release of silver ions as the silver pectate moiety disassociates. By adjusting the amount of the calcium pectate that is contained within the preparation one can achieve a well-controlled release of the silver ions at a rate that would be commensurate with the use of the silver ions as an antimicrobial agent and also in a concentration low enough to avoid too high a concentration of silver ions being achieved in a relatively short period of time which could possibly result in argyria.

Other ingredients within the composition so prepared as described above would be glycerol and carboxymethylcellulose in which these compositions are designed to enhance the tensile strength of a dressing so prepared, to ensure that such a dressing is nonbrittle, is sterilizable, and has a high level of wet-strength as well as resilience.

The inclusion of a surface active agent such as ammonium lauryl sulphate, and/or Tween 80 is designed to result in a homogenous composition of the various ingredients and ensure their stability.

In order to address the problem that many implants designed to treat a fracture by restricting movement of the fractured part, requires that any dressing attached to an implant, where the dressing containing an antimicrobial agent may have to be attached to a prosthetic device which has a relatively small circumference. Such a dressing should have excellent resilience and a degree of elasticity so that it does not fracture when subjected to the stress required by the implant.

Consequently, one of the salient advantages discovered in the preparation of the pectin compositions suitable for applications in implants utilizes the addition of sodium tetraborate to the pectin composition which enhances the elasticity of the dressing so prepared.

Another advantage of the invention described herein relates to the preparation of pectin compositions in which the aqueous portion of the composition can be removed by air-drying or regulated heat drying without the necessity of utilizing an expensive freeze-drying apparatus for its preparation.

Another salient advantage of the invention described herein concerns the feasibility of adding ingredients to the pectin composition, which ingredients may contain properties such as being particulate, having high viscosity, or having or resulting in a rheology which makes it undesirable or unfeasible for such compositions to be forced through a fine spinneret to produce the pectin fibers as currently practiced in U.S. Pat. No. 5,688,923.

Pectins can also be prepared with a degree of esterification (DE) which varies depending upon the esterification of the carboxyl groups of the pectin molecule. Since it is desirable in preparing a pectin moiety that it may be amenable to being precipitated with a 15 cation that cross-links with the pectin, then the degree of esterification should be minimized so that it should not interfere with the gelling reaction when certain cations are added to the pectin. The polyvalent cation for cross-linking the pectins is preferably a calcium ion. The DE of the pectin should be less than 50% so that a lower concentration of calcium and/or other polyvalent cations which precipitate the pectin may be utilized. In practice it has been found that the DE of pectins being less than 20% are the more desirable since lower calcium ion concentrations may be utilized for their gelation.

Pectins that are esterified can be converted to amidated pectins by reacting them with ammonium hydroxide as is well known in the profession, and such amidated pectins should preferably have a low degree of amidation in order to react with calcium or other cations which can precipitate the pectins as gels. The amidated pectins preferably should have a degree of amidation of approximately 25% for more optimum reaction with calcium ions in forming gels.

The antimicrobial moiety utilized for this patent is silver pectate. The silver ions will gradually be released from the silver pectate molecule from a dressing that has been prepared in which the silver pectate is a composition in a matrix. The silver ions have been shown in innumerable reports of dressings that are made with silver ions to have broad antimicrobial activity. In addition, there has not appeared any definitive evidence that microbial resistance to silver ions by microorganisms has taken place.

The concentration of silver ions will depend upon the application where the silver pectate dressing is to be either affixed to an implant, such as that used to fix a bone fracture which may require the release of antimicrobial activity for a 30-60 day period until the bones have appropriately healed, or where a catheter requires a coating of an antimicrobial agent to reduce the incidence of infections for a shorter period of use. The catheter as an implant may be inserted and maintained as such for a 24-36 hour period or perhaps longer. Consequently, the concentration of silver ions should be released from the matrix in which it is contained at a continuous rate, and the total amount contained therein can be adjusted in order to be specifically applicable for use with an implant that requires shorter or longer periods of implantation in humans or animals.

The inclusion within the matrix of a silver pectate molecule also includes a calcium pectate molecule which acts as an impediment to the migration of the silver ions to the surface of the dressing. As such it can be regulated to release a fixed amount over a relatively short or long period of time.

The use of desirable particulate matter such as micro-particles that can act as time-release particles, aqueous insoluble medicaments, or even the use of intact cells such as yeast cells, blood cells, or human or animal tissue cells, that might be desirable to apply with an implant to an open wound may be introduced into the silver pectate composition described herein. As is well known in the profession, the pH of the silver pectate composition may have to be adjusted to be commensurate with the cells that are to be incorporated into the final composition, and such pH adjustment is readily made by those skilled in the art. Such particulate matter and/or aqueous insoluble matter which can be incorporated into the silver pectate composition described in our patent is an attribute not feasible when pectin fibers are prepared by a spinning process.

Having set forth the tenets of the invention contained herein, the following non-limiting examples illustrate various compositions that are inherent in our invention.

### DESCRIPTION OF THE PREFERRED EMBODIMENTS

### EXAMPLE 1

Silver pectate, 8.5 grams is added to 600 ml of deionized water. The silver pectate may be prepared by reacting an aqueous soluble silver salt with an appropriate pectin. Examples of some soluble silver salts are: silver acetate, silver nitrate, silver sulfate, or silver fluogallate. The pectin utilized for the preparation of silver pectate should have a degree of esterification (DE) of less than 30% and a degree of amidation (DA) of less than 50%.

The silver pectate is dispersed by stirring and to this silver pectate suspension are added the following:
Maltodextrin . . . 4.5 gm
Glycerol . . . 21 ml
Carboxymethylcellulose . . . (low viscosity) 9 gm
Calcium gluconate . . . 2.4 gm.
Ammonium lauryl sulphate . . . 1.5 gm
Tween 80 . . . 6 ml

The above composition should be vigorously stirred until all of the ingredients have been dissolved and thoroughly mixed. The pectin composition thus prepared may contain a considerable amount of foam, which foam will not rise to the surface of the pectin composition, because the viscosity of the final pectin mixture is greater than the buoyancy of the foam. When poured onto a plate such as one made of plastic or metal, the dish containing the pectin mixture may be air-dried at 40° C. with overnight incubation, or placed into a drying oven with the following schedule of drying:
70° C.-2 hours
60° C.-2 hours
40° C.-until dry

### EXAMPLE 2

The composition as shown in Example 1 may also be dried by utilizing a 40° C. temperature for a 13-15 hour period (overnight).

### EXAMPLE 3

The pectin composition as prepared above in Example 1, when dried, may have a portion of it affixed to a metal implant to be utilized in the treatment of fractures. Such fixation of the dried pectin product may be affixed with the use of an adhesive suitable for implanting into a human or animal.

### EXAMPLE 4

The pectin composition as prepared in Examples 1 and 2 above, with continuous vigorous stirring has added to it 3.0 grams of sodium tetraborate (Na₂B₄O₇.10H₂O) dissolved in 50 ml of deionized water. The mixture is continuously stirred and then may be layered onto dishes for drying as described in Example 1 or Example 2 or in fact, after drying, may be attached to a suitable implant as described in Example 3.

### EXAMPLE 5

The ingredients that are described in Examples 1 to 3 may be prepared in the same way as described and to the semi-solid foamed composition is added 0.5 grams of ascorbic acid. The addition of ascorbic acid is designed to provide the beneficial effect of ascorbic acid as described in U.S. Pat. Nos. 5,177,065 and 4,778,679. After drying, it may be attached to a suitable implant as described in Examples 3 and 4 above.

### EXAMPLE 6

The pectin composition as described in Examples 1 to 5 inclusive above is prepared with stirring and to the final foamed pectin composition is added a dispersion of 10.0 ml of bovine collagen having a concentration of 100 mg per ml of distilled water. This composition can now be layered and dried, or adhered to a prosthetic for implantation in humans or animals as described in Examples 1 to 5 above.

### EXAMPLE 7

The pectin composition as described in Examples 1 to 6 above, may have had added to it desirable components of particulate products. Such particulate products may be antimicrobial in nature, agents to enhance tissue regeneration, and/or particles of bone to enhance healing of a fracture. A dressing prepared as described herein and dried may then be affixed to an implant as described in Examples 3 or 4. Sterilization of such composition may be achieved utilizing ethylene oxide.

The above descriptions and examples illustrate particular constructions including the preferred embodiments of the solutions. However, the invention is not limited to the precise constructions describer herein, but, rather, all modifications and improvements thereof encompassed within the scope of the invention.

Many of the examples described herein utilize the surface active agents such as those characterized as Tween 80 or Pluronic L64. These surface-active agents are utilized primarily to effect a dispersion between the non-aqueous miscible components utilized in achieving a coercive mixture with the aqueous soluble pectins in order to ensure a homogeneity throughout the solutions that are then precipitated as insoluble pectate compositions.

These surface active agents are also utilized in order to improve the wetting of a medical dressing or bandage in the event that a wound may be exudating, and the enhanced wicking in such a bandage or medical dressing serves to quickly absorb any blood or serum from a wound site into the dressing. Other surface active agents, such as the Na salt of dodecyl SO₄ (sodium lauryl sulfate) or a member of the group of Tweens: (Tween 20, polyoxyethylene sorbitan monolaurate; Tween 40, polyoxyethylene sorbitan monopalmitate; or Tween 85, polyoxyethylene sorbitan trioleate may be incorporated into the pectin composition without deviating from the novelty of the invention described herein.

In the examples cited herein, calcium gluconate has been utilized to provide the calcium ions which precipitate the aqueous insoluble calcium pectate which insolubilization may also serve to entrap into the calcium pectate matrix other components as described herein. It is clear, as has been mentioned, that other salts may be utilized to precipitate the pectins such as those of aluminum, zinc, copper, chromium, or silver and these insoluble pectates may be readily utilized to precipitate the various mixtures described in the examples provided herein without deviating from the essential merits of this invention. However, if the pectate compositions are to be utilized in or on biological tissues, or as implants, the particular salt utilized to precipitate the pectins should be dictated by any restraints of toxicity or other untoward reactions that might results from their use for the preparation of surgical implants.

In the examples cited herein, bovine collagen has been utilized to provide a haemostatic agent in the event of frank bleeding of a wound. It is clear, that collagens other than bovine collagen may be utilized for this purpose such as porcine collagen or human collagen without deviating from the essential merits of this invention.

In the examples cited, the pectin had a degree of esterification of less than 30% in order to achieve a relatively high degree of reactivity with calcium and other ions. It is clear however, that pectins other than those having a degree of esterification of less than 30% may be utilized without deviating from the essential tenets of the invention.

In the examples cited, the pectin had a degree of amidation of less than 50%. It is clear however, that pectins other than those having a degree of amidation of less than 50% may be utilized without deviating from the essential tenets of the invention.

### REFERENCES CITED

Anwar, Hosmin et al. “Tobramycin resistance of mucoid Pseudomonas aeruginosa biofilm grown under ion limitation,” Journal of Antimicrobial Chemotherapy (1989) 24, 647-655
Azary Technologies LLC 510(k) K040518
Blom, A. W. “Infection after total knee arthroplasty,” The Journal of Bone and Joint Surgery 2004;86-B, No. 5, 688-91, July 2004
Fishman, Marshall L. and Joseph Jen, Ed., Chemistry and Function of Pectins, American Chemical Society 1986
Goodman, L. and A. Gilman (1965 The Pharmacological Basis of Therapeutics, 3^{rd} Ed. New York, The Macmillan Company)
Howmedica International S. de R. L.
Illingworth, et al. (J. Heart Valve Dis. 1998 September; 7(5): 524-30)
Lawrence and Block, Disinfection, Sterilization and Preservation. Chapters 24 and 28, Lea and DeBig, Philadelphia, 1968
McDowell, R. H., Properties of Alginates,—Alginate Industries, Limited, London, England 1961
The Merck Index, 7^{th} Edition. Stecher, P. G., Ed. Rahway, N. J. Merck & Co., Inc., 1960.
Minelli, et al. (J. Antimicrob. Chemother. 2004 August; 54(2):570; author reply 570-1)
Moojen, et al. (J. Arthroplasty. 2008 December; 23(8):1152-6)
New England Journal of Medicine (2004:350-1422-9) 33
Nickel, J. C. et al. Tobramycin resistance of Pseudomonas aeruginosa cells growing as a biofilm on urinary catheter material. Journal of Antimicrobial Agents Chemotherapy 1985; 27(4): 619-24
Nussinovitch, A. Hydrocolloid Applications. Published by Blackie Academic & Professional, 1997, Chapter 5—Pectins
Rochester Medical 510(k) K033477
Scardillo, Judith. Infection Control Resource, Vol. 2 No. 1, pp. 2-7—Saxe Healthcare Communications, Burlington, Vt.
Spectrum Laboratory Products, Inc. Gardena, Calif. MSDS Sheet, Sep. 13, 2006
Supfle, K. and R. Werner, (1951 Microdosimetric investigation of the olygodynamic effect of silver. Mikrochemie ver. Mikrochim. Acta, 36/37, 866-881)
Trop, Marija, et al. Silver-Coated Dressing Acticoat Caused Raised Liver Enzymes and Argyria-like Symptoms in Burn Patient. The Journal of TRAUMA Injury, Infection, and Critical Care Volume 60-Number 3: 648-652, March 2006
TyRx Pharma, Inc. 510(k) Number 063091

### U.S. Patents

| | | |
|---|---|---|
| 2,674,619 | Apr. 6, 1954 | Lundsted |
| 5,147,648 | Sep. 15, 1992 | Bannert |
| 4,778,679 | Oct. 18, 1988 | Silvetti |
| 5,177,065 | Jan. 5, 1993 | Silvetti, Sr., et al. |
| 5,674,524 | Oct. 7, 1997 | Scherr |
| 5,718,916 | Feb. 17, 1998 | Scherr |
| 7,128,929 | Oct. 31, 2006 | Scherr |
| 6,696,077 | Feb. 24, 2004 | Scherr |
| 3,639,575 | Feb. 1, 1972 | Schmolka |
| 4,184,974 | Jan. 22, 1980 | Van Leuven |
| 5,688,923 | Nov. 18, 1997 | Gerrish, et al.. |

### Foreign Patents

| | | |
|---|---|---|
| 221859 (India) | Jul. 8, 2008 | Scherr |
| 2,357,765 (Great Britain) | Apr. 21, 2004 | Scherr |
| 2322266 (Russia) | Feb. 18, 2003 | Scherr |

I claim:

1. The process for making a cation cross-linked pectin composition comprising:
a) Cross-linking the pectin with an aqueous soluble silver salt in distilled or deionized water and,
b) with continuous stirring, adding to the silver pectate suspension, a plasticizing agent and,
c) a surface active agent.

2. The process of claim 1 wherein a non-pectin polysaccharide is added to the silver pectate composition.

3. The process of claim 2 wherein the non-pectin polysaccharide is selected from the group consisting of carboxymethylcellulose, carboxymethyl ethyl cellulose, hyaluronic acid, carrageenan, and/or gellan gum.

4. The process of claim 3 wherein the non-pectin polysaccharide is carboxymethylcellulose.

5. The process of claim 1 where the aqueous soluble silver salt may be silver acetate, silver fluogallate, silver nitrate, and/or silver sulfate.

6. The process of claim 1 wherein a cation is selected from a metal ion derived from salts selected from the group consisting of alkaline earth metal salts, alkali metal salts, transition metal salts and/or mixtures thereof, and added to the composition of claim 1.

7. The process of claim 6 wherein the cation is selected from a group consisting of calcium, barium, magnesium, zinc, iron, aluminum, copper, strontium, manganese, and/or mixtures thereof.

8. The process of claim 1 wherein the upper limit of the DE of the pectin that is cross-linked with the silver is 50%.

9. The process of claim 1 wherein the upper limit of the DE of the pectin that is cross-linked with the silver is 30%.

10. The process of claim 1 wherein the upper limit of the DE of the pectin that is cross-linked with the silver is 5%.

11. The process of claim 1 wherein the lower limit of the DE of the pectin that is cross-linked with the silver is 0%.

12. The process of claim 1 wherein an aqueous soluble calcium salt is added to the silver pectate composition.

13. The process of claim 12 wherein the aqueous soluble calcium salt added to the silver pectate composition may be calcium gluconate, calcium chloride, calcium chlorate, calcium lactate, calcium nitrate, calcium propionate, and/or calcium thiosulfate.

14. The process of claim 13 wherein the aqueous soluble calcium salt is calcium gluconate.

15. The process of claim 1 wherein a surface active agent may be selected from the group ammonium lauryl sulphate, Tween 80, and/or Tween 20.

16. The process of claim 15 wherein Tween 80 is added to the silver pectate composition.

17. The process of claim 1 where the surface active agent ammonium lauryl sulphate is added to the silver pectate composition.

18. The process of claim 1 where glycerol is added to the silver pectate composition.

19. The process of claim 1 wherein the upper limit of the DA of the calcium sensitive pectin is 25%.

20. The process of claim 1 wherein the upper limit of the DA of the calcium sensitive pectin is 20%.

21. The process of claim 1 wherein the lower limit of the DA of the calcium sensitive pectin is 0%.

22. The process of claim 1 wherein the lower limit of the DA of the calcium sensitive pectin is 5%.

23. The process of claim 1 wherein the lower limit of the DA of the calcium sensitive pectin is 10%.

24. The process of any one of the previous claims 1 to 23 inclusive, in which the dried silver pectate composition is affixed to an implant utilized for the correction of fractures in animals or man.

25. The process of any one of the previous claims 1 to 23 inclusive, in which the dried silver pectate composition is affixed to an implant utilized for the correction of pathological conditions of bones and/or tissues in man and animals.

26. The process for making a cation cross-linked pectin composition comprising:
a) Cross-linking the pectin with an aqueous soluble silver salt in distilled or deionized water and,
b) with continuous stirring, adding to the silver pectate suspension, a plasticizing agent and,
c) a surface active agent and,
d) a medicament.

27. The process of claim 26 wherein a non-pectin polysaccharide is added to the silver pectate composition.

28. The process of claim 26 wherein the non-pectin polysaccharide is selected from the group consisting of carboxymethylcellulose, carboxymethyl ethyl cellulose, hyaluronic acid, carrageenan, and/or gellan gum.

29. The process of claim 28 wherein the non-pectin polysaccharide is carboxymethylcellulose.

30. The process of claim 26 where the aqueous soluble silver salt may be silver acetate, silver fluogallate, silver nitrate, and/or silver sulfate.

31. The process of claim 26 wherein a cation is selected from a metal ion derived from salts selected from the group consisting of alkaline earth metal salts, alkali metal salts, transition metal salts and/or mixtures thereof, and added to the composition of claim 26.

32. The process of claim 31 wherein the cation is selected from a group consisting of calcium, barium, magnesium, zinc, iron, aluminum, copper, strontium, manganese, and/or mixtures thereof.

33. The process of claim 26 wherein the upper limit of the DE of the pectin that is cross-linked with the silver is 50%.

34. The process of claim 26 wherein the upper limit of the DE of the pectin that is cross-linked with the silver is 30%.

35. The process of claim 26 wherein the upper limit of the DE of the pectin that is cross-linked with the silver is 5%.

36. The process of claim 26 wherein the lower limit of the DE of the pectin that is cross-linked with the silver is 0%.

37. The process of claim 26 wherein an aqueous soluble calcium salt is added to the silver pectate composition.

38. The process of claim 37 wherein the aqueous soluble calcium salt added to the silver pectate composition may be calcium gluconate, calcium chloride, calcium chlorate, calcium lactate, calcium nitrate, calcium propionate, and/or calcium thiosulfate.

39. The process of claim 38 wherein the aqueous soluble calcium salt is calcium gluconate.

40. The process of claim 26 wherein a surface active agent may be selected from the group ammonium lauryl sulphate, Tween 80, and/or Tween 20.

41. The process of claim 40 wherein Tween 80 is added to the silver pectate composition.

42. The process of claim 26 where the surface active agent ammonium lauryl sulphate is added to the silver pectate composition.

43. The process of claim 26 where glycerol is added to the silver pectate composition.

44. The process of claim 26 wherein the upper limit of the DA of the calcium sensitive pectin is 25%.

45. The process of claim 26 wherein the upper limit of the DA of the calcium sensitive pectin is 20%.

46. The process of claim 26 wherein the lower limit of the DA of the calcium sensitive pectin is 0%.

47. The process of claim 26 wherein the lower limit of the DA of the calcium sensitive pectin is 5%.

48. The process of claim 26 wherein the lower limit of the DA of the calcium sensitive pectin is 10%.

49. The process of any one of the previous claims 26 to 48 inclusive, in which the dried silver pectate composition is affixed to an implant utilized for the correction of fractures in animals or man.

50. The process of any one of the previous claims 26 to 48 inclusive, in which the dried silver pectate composition is affixed to an implant utilized for the correction of a pathological condition of bones and/or tissues in man and animals.

51. The process for making a cation cross-linked pectin composition comprising:
a) Cross-linking the pectin with an aqueous soluble silver salt in distilled or deionized water and,
b) with continuous stirring, adding to the silver pectate suspension, a plasticizing agent and,
c) a surface active agent and,
d) particulate agents.

52. The process of claim 51 wherein a non-pectin polysaccharide is added to the silver pectate composition.

53. The process of claim 52 wherein the non-pectin polysaccharide is selected from the group consisting of carboxymethylcellulose, carboxymethyl ethyl cellulose, hyaluronic acid, carrageenan, and/or gellan gum.

54. The process of claim 53 wherein the non-pectin polysaccharide is carboxymethylcellulose.

55. The process of claim 51 where the aqueous soluble silver salt may be silver acetate, silver fluogallate, silver nitrate, and/or silver sulfate.

56. The process of claim 51 wherein a cation is selected from a metal ion derived from salts selected from the group consisting of alkaline earth metal salts, alkali metal salts, transition metal salts and/or mixtures thereof, and added to the composition of claim 51.

57. The process of claim 56 wherein the cation is selected from a group consisting of calcium, barium, magnesium, zinc, iron, aluminum, copper, strontium, manganese, and/or mixtures thereof.

58. The process of claim 51 wherein the upper limit of the DE of the pectin that is cross-linked with the silver is 50%.

59. The process of claim 51 wherein the upper limit of the DE of the pectin that is cross-linked with the silver is 30%.

60. The process of claim 51 wherein the upper limit of the DE of the pectin that is cross-linked with the silver is 5%.

61. The process of claim 51 wherein the lower limit of the DE of the pectin that is cross-linked with the silver is 0%.

62. The process of claim 51 wherein an aqueous soluble calcium salt is added to the silver pectate composition.

63. The process of claim 62 wherein the aqueous soluble calcium salt added to the silver pectate composition may be calcium gluconate, calcium chloride, calcium chlorate, calcium lactate, calcium nitrate, calcium propionate, and/or calcium thiosulfate.

64. The process of claim 63 wherein the aqueous soluble calcium salt is calcium gluconate.

65. The process of claim 51 wherein a surface active agent may be selected from the group ammonium lauryl sulphate, Tween 80, and/or Tween 20.

66. The process of claim 65 wherein Tween 80 is added to the silver pectate composition.

67. The process of claim 51 where the surface active agent ammonium lauryl sulphate is added to the silver pectate composition.

68. The process of claim 51 where glycerol is added to the silver pectate composition.

69. The process of claim 51 wherein the upper limit of the DA of the calcium sensitive pectin is 25%.

70. The process of claim 51 wherein the upper limit of the DA of the calcium sensitive pectin is 20%.

71. The process of claim 51 wherein the lower limit of the DA of the calcium sensitive pectin is 0%.

72. The process of claim 51 wherein the lower limit of the DA of the calcium sensitive pectin is 5%.

73. The process of claim 51 wherein the lower limit of the DA of the calcium sensitive pectin is 10%.

74. The process of any one of the previous claims 51 to 73 inclusive, in which the dried silver pectate composition is affixed to an implant utilized for the correction of fractures in animals or man.

75. The process of any one of the previous claims 51 to 73 inclusive, in which the dried silver pectate composition is affixed to an implant utilized for the correction of a pathological condition of bones and/or tissues in man and animals.

76. The process of claim 51 in which the particulate agents may be cellular, antimicrobial, agents to enhance tissue regeneration, and/or particles of bone to enhance healing of fracture.

77. The process of claim 76 in which the cellular material may be microbial or human cellular material.

78. The process of claim 76 in which the antimicrobial agents may be antibacterial, antimycotic, and/or antiviral.

79. The process of claim 76 in which the particulate agents to enhance tissue regeneration are selected to enhance bone tissue regeneration and/or soft tissue regeneration.

80. The composition of claims 1 to 79 inclusive wherein is added the chemotactic agent maltodextrin.

81. The composition of claims 1 to 80 wherein is added the agent collagen.

82. The composition of claims 1 to 81 wherein the collagen is bovine collagen.

83. The composition of claims 1 to 81 wherein the collagen is porcine collagen.

84. The composition of claims 1 to 81 wherein the collagen is human collagen.

85. The process of any one of the previous claims 26 to 84 inclusive, in which the dried silver pectate composition is affixed to an implant utilized for the correction of fractures in animals or man.

86. The process of any one of the previous claims 26 to 84 inclusive, in which the dried silver pectate composition is affixed to an implant utilized for the correction of pathological conditions of bones and/or tissues in man and animals.

87. The process of any one of the previous claims 1 through 84 inclusive in which prosthetic implants such as catheters and pacemakers are coated with the composition of claims 1 to 84 inclusive.

88. The composition of any one of the previous claims 1 through 87 which is sterile.

89. The composition of any one of the previous claims 1 through 87 which is sterilized by ethylene oxide.

90. The composition of any one of the previous claims 1 through 87 which is sterilized by ionizing radiation.

91. The composition of any one of the previous claims 1 through 87 which is sterilized by chemical preservatives.

92. A silver pectate moiety.
